(19) United States
(12) Patent Application Publication
(10) Pub. No.: US 2008/0195068 A1
(43) Pub. Date: Aug. 14, 2008

(54) SANITARY NAPKIN

(76) Inventor: Wan-Sheng Kao, Taipei (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 11/672,644

(22) Filed: Feb. 8, 2007

(51) Int. Cl. A61F 13/15

(52) U.S. Cl. 604/361; 604/378; 604/385.101; 604/385.01

(57) ABSTRACT

A sanitary napkin has a top layer, an absorbent layer and a bottom layer. The absorbent layer has a hollow portion with at least one isolating layer. The isolating layer, the top layer and the bottom layer have multiple ventilating holes corresponding to the hollow portion. Irregular absorbent material with voids is disposed between two adjacent isolating layers to allow air convection to disperse heat, so that the menstrual blood is quickly drained and absorbed by the absorbent layer to keep the sanitary napkin dry at the top layer and to avoid toxic vapors that might enter the vagina or the uterus and thus incur pathological changes. The toxic vapors are caused by chemical qualitative change if menstrual blood with high heat is retained on the top layer. Therefore, the sanitary napkin is safe and comfortable in use.

### BACKGROUND OF THE INVENTION

1. Field of the Invention

The present invention relates to a sanitary napkin, and more particularly to a sanitary napkin that makes the menstrual blood dispersed and absorbed therein quickly and ventilative in use.

2. Description of Related Art

Females at the age of puberty have sexual developments such as uterus maturation that causes menses at normally monthly intervals. During menstruation, uterine intima is stimulated by estrogen, gets thick and then degenerates to separate from the uterus and to be discharged via the vaginal opening with menstrual blood. Menses is a particular sexual feature to the females and causes troubles to the females since ancient age.

According to historical papers, women in age of cave dwelling use sponges or hay to absorb the menstrual blood. Some women in ancient Egypt use softened Egyptian paper reed to make absorbent strips and some Greek women use linen wrapping around small sticks to make absorbent devices for menses treatment or take clean cotton to make pads that can be reused after washing. In China, women put an elongated clean cloth at the crotch to absorb the menstrual blood. After use, the clean cloth is washed with water and alum for reuse. Because the menses cause many troubles, women do not go on a long journey in ancient age during menstruation.

In the beginning of twentieth century, some companies manufacture napkin products. One conventional napkin product was an absorbent sheet attached on an undergarment by safety pins. Another conventional napkin product was a menstrual pad composed of the absorbent sheet with multiple belts to tie on the waist of a user. However, the menstrual pad is still inconvenient in use. Until 1921, Kimberly Clark Company manufactured a first disposable sanitary napkin that was the original model of all various sanitary napkin products using now. Thereby, women are not bothered by the inconvenience of the menstrual problems anymore.

A conventional napkin has a top layer made of soft cotton textile, an absorbent core made of board pulp embedded inside the conventional napkin and an anti-leakage membrane attached between the top layer and the absorbent core. The conventional napkin further has a glue layer formed at a bottom and two wings extending from sides to keep the napkin from sliding on the undergarment so that the menstrual blood can be completely absorbed by the absorbent core.

Although the conventional napkin above provides a solution for menstrual problems, some drawbacks of the conventional napkin still exist. Because the menstrual blood flows randomly, the female user feels uncomfortable about directly contacting the warm menstrual blood especially when the menstrual blood is plenty. To this drawback, manufacturers developed another conventional napkin that has the absorbent core shaped into a central bulge and two side low portions in accordance with the crutch to make the menstrual blood been absorbed well when the menstrual blood just comes out of the vaginal opening. However, the menstrual blood is warm and substantially gathered at the central bulge that directly abuts the crutch to cause an uncomfortable burning feeling to the female user. When the central bulge abuts tightly against the crutch, the menstrual blood can not be absorbed quickly. Moreover, heat of the menstrual blood causes trace vaporization of polyvinyl chloride monomer that is toxic, enters the vagina and causes some pathological changes at the cervix.

### SUMMARY OF THE INVENTION

One main objective of the present invention is to provide a sanitary napkin that makes the menstrual blood quickly dispersed in an absorbent material and away from the crotch to avoid uncomfortable feeling so that the menstrual blood does not cause any toxic vapor due to heating to the absorbent material.

Another main objective of the present invention is to provide a sanitary napkin that has multiple ventilating holes and a hollow portion for conducting the menstrual blood quickly and an isolating layer to make the menstrual blood been absorbed by the absorbent material. By further having multiple holes in a bottom layer, multiple ventilating holes in a covering layer, and the isolating layer, the sanitary napkin has convection with air outside and is ventilative to keep the crutch dry, crisp and comfortable.

Still another main objective of the present invention is to provide a sanitary napkin that has an opening defined in a bottom layer and a sensor layer partially attached at the opening to indicate a changing occasion by color changes after the sensor layer is stained with the menstrual blood.

To achieve the foregoing objective, the sanitary napkin comprises a top layer, an absorbent layer and a bottom layer laminated in sequence, wherein improvements of the sanitary napkin comprise:

a hollow portion is defined in the top layer and through the absorbent layer to the bottom layer along a central line; and

at least one isolating layer accommodated inside the hollow portion.

Thereby, the sanitary napkin has excellent absorbent efficiency and always keeps dry and cool at the top layer.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a sanitary napkin in accordance with the present invention;

FIG. 2 is a cross-sectional side view of a first embodiment of the sanitary napkin along line A-A in FIG. 1;

FIG. 3 is a cross-sectional side view of a second embodiment of the sanitary napkin along line A-A in FIG. 1;

FIG. 4 is a cross-sectional side view of a third embodiment of the sanitary napkin along line A-A in FIG. 1;

FIG. 5 is a top view of an isolating layer of the sanitary napkin;

FIG. 6 is an operational view of the sanitary napkin; and

FIG. 7 is a bottom view of the sanitary napkin in FIG. 1.

### DETAILED DESCRIPTION OF THE PREFERRED EMBODIMENT

A sanitary napkin in accordance with the present invention comprises a top layer, an absorbent layer and a bottom layer. The absorbent layer has a hollow portion with at least one isolating layer. The isolating layer, the top layer and a bottom layer have multiple ventilating holes corresponding to the hollow portion. Irregular absorbent material with voids is disposed between two adjacent isolating layers to allow air convection to disperse heat so that the menstrual blood is drained quickly and absorbed by the absorbent layer to keep the sanitary napkin dry at the top layer and to avoid toxic vapor that might enter the vagina or the uterus and thus incur pathological changes, wherein the toxic vapor is caused by chemical qualitative change if the menstrual blood with high heat is retained on the top layer.

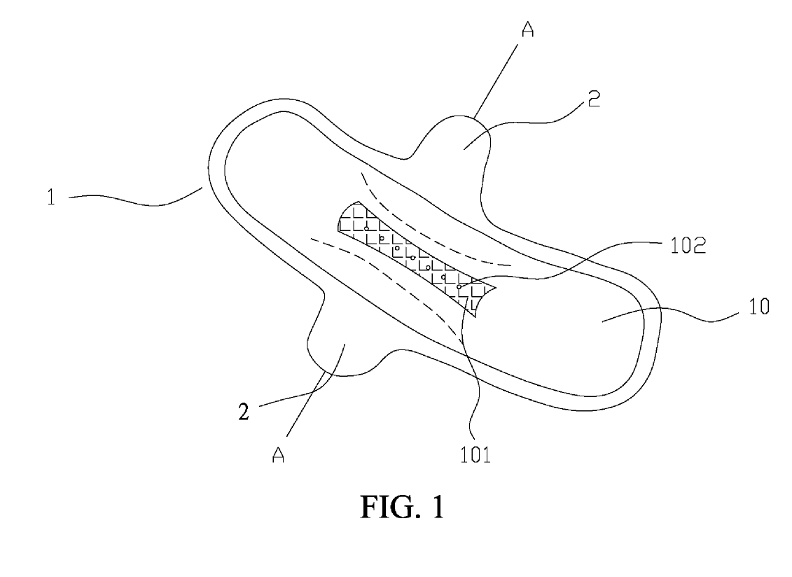

With reference to FIG. 1, a preferred embodiment of the sanitary napkin comprises a pad body 1, two wings 2 formed at two sides of the pad body 1, a top layer 10, a cover layer 101 and multiple ventilating holes 102 in the cover layer 101, wherein the multiple holes 102 is formed at a center transversal line of the pad body 1.

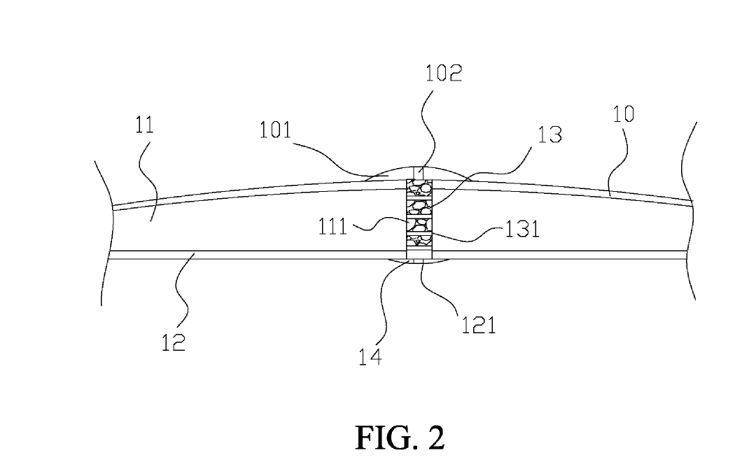

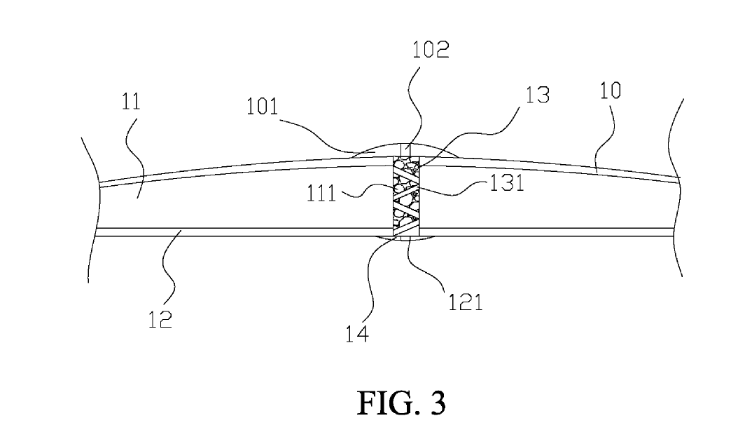

As shown in FIGS. 2 and 3, the pad body 1 comprises the top layer 10, an absorbent layer 11 and a bottom layer 12. The top layer 10 is made of silk cloth that is smooth in texture and super-ventilative to make contact gentle and comfortable without muggy feeling. The absorbent 11 layer is made of 100% pure ceiba pulp or a mixture of macromolecular polymers to quickly absorb warm menstrual blood and eliminate thermal and unpleasant odors. The bottom layer 12 is made of super-thin plastic membrane having anti-leakage efficiency and has two glue strips 121 attached on an outer side of the bottom layer 12 for attaching convenience.

Especially, the whole pad body 1 has an elongated hollow portion 13 defined along the center transversal line from the top layer 10 to the bottom layer 12 inside the pad body 1 to correspond to the crutch and to facilitate the menstrual blood to disperse downward. The cover layer 101 made of silk covers an opening of the hollow portion 13 on the top layer 10 and has the multiple ventilating holes 102 defined in the cover layer 101 and corresponding to hollow portion 13 to facilitate the drain of the menstrual blood and to make the sanitary napkin ventilative. Moreover, the hollow portion 13 has multiple isolating layers 131 in parallel arrangement or continuous slopes arrangement and is filled with irregular or regular absorbent bodies 111 between the multiple isolating layers 131, the top layer 10 and the bottom layer 12. Particularly, the isolating layers 131 are made of absorbent material, accommodated inside the hollow portion 13 and connected to the absorbent layer to absorb and guide the menstrual blood to the absorbent layer.

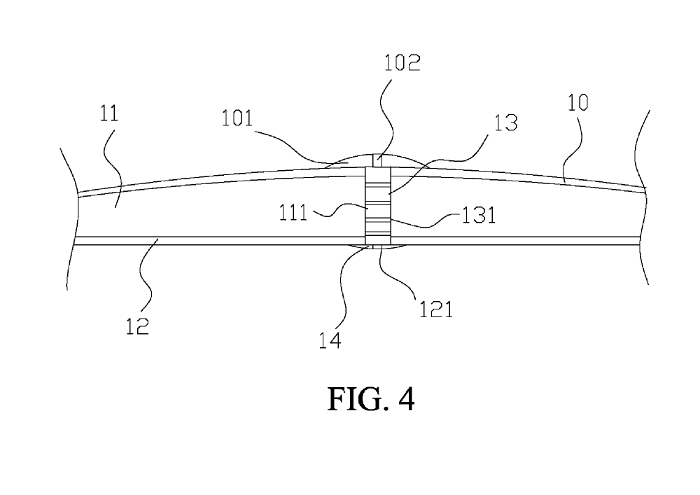

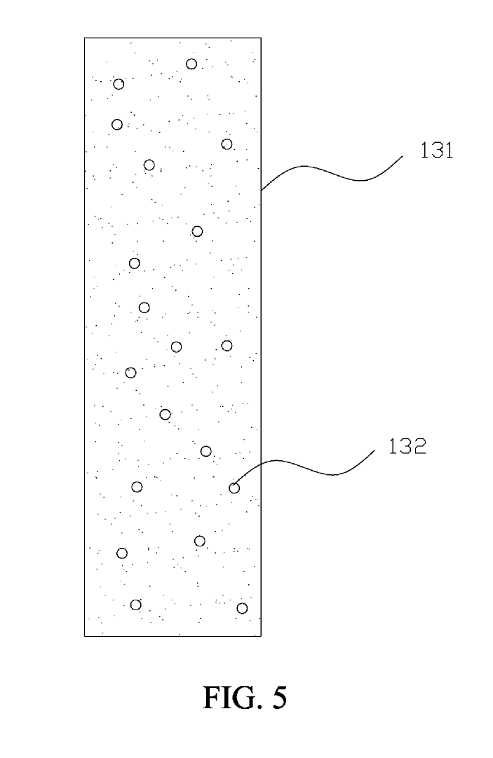

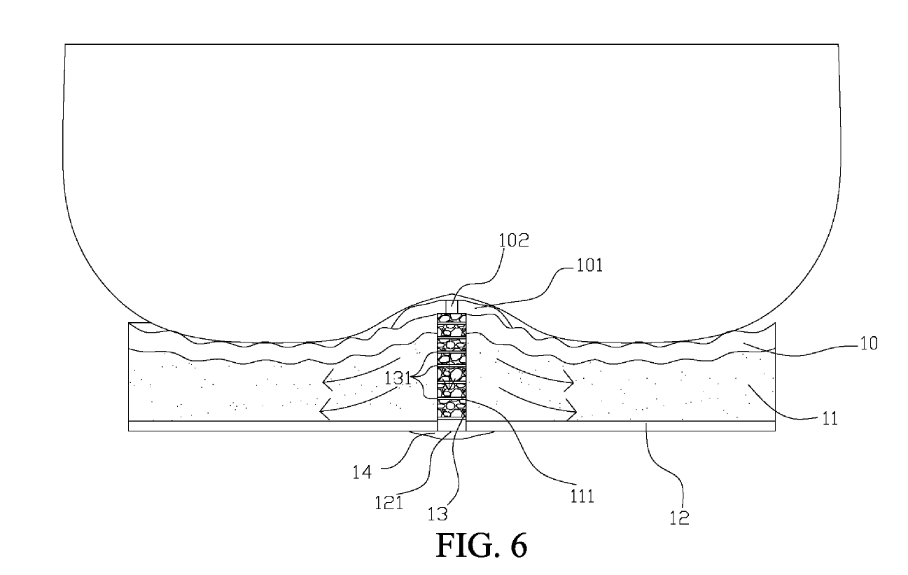

As shown in FIG. 6, by having the foregoing structure, the menstrual blood drained into the hollow portion 13 is conducted to the absorbent layer 11 around by the isolating layer 131 and absorbed by the absorbent bodies 111 between the isolating layers 131. As shown in FIG. 5, each isolating layer 131 being an elongated strip has absorbing and conducting efficiency to the menstrual blood and further has multiple optional through hole 132 to drain the menstrual blood downward. Moreover, the multiple isolating layers 131 is selectively and immovably attached to the top layer 10, the absorbent layer 11 and the bottom layer 12 or is movably enclosed by the top layer 10, the absorbent layer 11 and the bottom layer 12 such as a folded isolating layer 131 is filled into the hollow portion 13 (as shown in FIG. 4), wherein the multiple isolating layers has intervals therebetween.

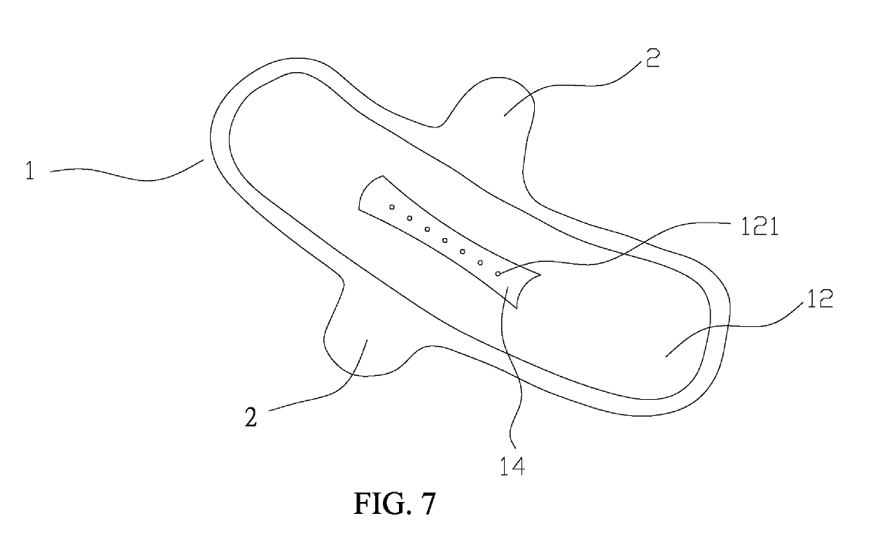

As shown in FIG. 7, the bottom layer 12 of the sanitary napkin 1 has an opening defined corresponding to the hollow portion 13, wherein the opening can be modified into multiple apertures 121 and be covered with a sensor layer 14. When the menstrual blood contacts the sensor layer 14, color of the sensor layer 14 varies that means the sanitary napkin should be changed so that the crutch is kept crispy and has no pigmentations or sanitary problems caused by the damp of the menstrual blood.

As shown in FIG. 6, when the sanitary napkin is attached to the undergarment, the hollow portion 13 aligns to the vagina opening so that the menstrual blood quickly flows into the hollow portion 13 via the ventilative holes 102 on the cover layer 101 through the top layer 10. Meanwhile, the multiple isolating layers 131 keep the menstrual blood from reflowing back to the top layer 10 and then conduct the menstrual blood to quickly be dispersed to the absorbent layer 11 around and to be absorbed by the absorbent bodies 111 between the multiple isolating layers 131. Thereby, the menstrual blood can be quickly absorbed by the pad body 1 of the sanitary napkin and the crutch is kept dry and clean all the time to make the user feel comfortable. When the menstrual blood is plenty, the sensor layer 14 on the bottom layer 12 shows color changes to indicate that the sanitary napkin should be changed because the absorbent layer 11 is saturated, wherein the user observes the color changes via the bottom of the panty.

According to above description, the sanitary napkin has the following advantages:

1. Because the sanitary napkin has the hollow portion 13 at the central transversal line, the menstrual blood can quickly flows into the hollow portion 13 and the isolating layers 131 with the absorbent bodies 111 conduct the menstrual blood to quickly disperse to the absorbent layer 11 so that the top layer 10 is kept dry.

2. Heat of the menstrual blood is quickly reduced after dispersing to the absorbent layer 11. Therefore, chemical reaction does not happen when the heat is low and no toxic gas will be generated so that possible pathological changes are eliminated.

3. The ventilating holes 102 on the cover layer 101, the through holes in the isolating layers and the apertures in the bottom layer altogether provide ventilation efficiency to avoid damp so as to reduce the uneasy feeling when the female uses the sanitary napkin.

Although this invention has been described in its preferred form with a certain degree of particularity, it is understood that the present invention of the preferred form has been made only by way of example and that numerous changes in the details of construction and the combination and arrangement of parts any be resorted to without departing from the spirit and scope of the invention.

1. A sanitary napkin comprising a top layer, an absorbent layer and a bottom layer laminated in sequence, wherein:
a hollow portion is defined in the top layer and through the absorbent layer to the bottom layer along a central line; and
multiple isolating layers are accommodated inside the hollow portion and have multiple absorbent bodies clamped between two adjacent isolating layers with voids to improve air ventilation.

2. The sanitary napkin as claimed in claim 1, wherein the multiple isolating layers have absorbing and conducting efficiency and are arranged parallel to the bottom layer.

3. The sanitary napkin as claimed in claim 1, wherein the multiple isolating layers have absorbing and conducting efficiency and are arranged in a zigzag pattern, with the multiple isolating layers having ends extending between the hollow portion at oblique angles.

4. The sanitary napkin as claimed in claim 1, wherein each of the multiple isolating layers has multiple through holes.

5. The sanitary napkin as claimed in claim 1, wherein the multiple isolating layers are immovably attached to the top layer, the absorbent layer and the bottom layer.

6. The sanitary napkin as claimed in claim 1, wherein the multiple isolating layers are movably attached to the top layer, the absorbent layer and the bottom layer.

7. The sanitary napkin as claimed in claim 1, wherein the top layer has multiple ventilating holes aligned with the hollow portion.

8. (canceled)

9. The sanitary napkin as claimed in claim 1, wherein the absorbent layer is made of pure ceiba pulp.

10. The sanitary napkin as claimed in claim 1, wherein the absorbent layer is made of macromolecule polymer.

11. The sanitary napkin as claimed in claim 1, wherein the bottom layer has an opening defined in the bottom layer aligned with the hollow portion; with the sanitary napkin further comprising a sensor attached to the bottom layer and partially covering the opening of the bottom layer, wherein the sensor changes colors.
